(19) United States
(12) Patent Application Publication
(10) Pub. No.: US 2022/0178824 A1
(43) Pub. Date: Jun. 9, 2022

(54) METHOD FOR DETECTING HEAVY METAL POLLUTANTS USING A FLUORESCENT MATERIAL FROM BACILLUS ENDOPHYTICUS AND METHOD FOR MAKING

(71) Applicant: Imam Abdulrahman Bin Faisal University, Dammam (SA)

(72) Inventors: Mahmoud Mohamed BEREKAA, Dammam (SA); Mohamed Farouk EZZELDIN, Dammam (SA)

(73) Assignee: Imam Abdulrahman Bin Faisal University, Dammam (SA)

(21) Appl. No.: 17/115,183

(22) Filed: Dec. 8, 2020

(51) Int. Cl. G01N 21/64; C12N 1/20

(52) U.S. Cl. CPC G01N 21/643; C12N 2523/00; C12N 1/20; G01N 21/6447

(57) ABSTRACT

A method for detecting heavy metals using a fluorescent material produced from Bacillus endophyticus such as strain DS43 which is a wild-type or natural isolate from soil in Dammam City, Saudi Arabia. The fluorescent material exhibits fluorescence under UV irradiation at a wavelength of approximately 365 nm which disappears after exposure to heavy metals. A method for culturing Bacillus endophyticus producing the fluorescent material and to methods for extracting this material for use in detecting heavy metals.

### BACKGROUND OF THE INVENTION

### Field of the Invention

The invention falls within the fields of environmental biotechnology and specifically concerns production of a fluorescent material from Bacillus endophyticus and its use in detecting heavy metal pollutants.

### Description of Related Art

With increasing industrialization and development of industries that mine, process, or use heavy metals, the risk of heavy metal waste water pollution has become increasingly serious. Waste water contaminated with heavy metals not only causes serious damage to the human body, but also destroys the environment.

Chromium, arsenic, cadmium, mercury, and lead have the greatest potential to cause harm on account of their extensive use, the toxicity of some of their combined or elemental forms, and their widespread distribution in the environment. Hexavalent chromium, for example, is highly toxic as are mercury vapor and many mercury compounds. These five elements have a strong affinity for sulfur; in the human body they usually bind, via thiol groups (—SH), to enzymes responsible for controlling the speed of metabolic reactions. The resulting sulfur-metal bonds inhibit the proper functioning of the enzymes involved; human health deteriorates, sometimes fatally. Chromium (in its hexavalent form) and arsenic are carcinogens; cadmium causes a degenerative bone disease; and mercury and lead damage the central nervous system.

Lead is the most prevalent heavy metal contaminant. Other heavy metals noted for their potentially hazardous nature, usually as toxic environmental pollutants, include manganese (central nervous system damage); cobalt and nickel (carcinogens); copper, zinc, selenium and silver (endocrine disruption, congenital disorders, or general toxic effects in fish, plants, birds, or other aquatic organisms); tin, as organotin (central nervous system damage), antimony (a suspected carcinogen); and thallium (central nervous system damage).

Heavy metals can degrade air, water, and soil quality, and subsequently cause health issues in plants, animals, and people, when they become concentrated as a result of industrial activities. Common sources of heavy metals in this context include mining and industrial wastes; vehicle emissions; lead-acid batteries; fertilizers; paints; and treated timber; aging water supply infrastructure; and microplastics floating in the world's oceans.

Conventional techniques for detecting and measuring heavy metal contamination are often complicated, slow, expensive, or require genetic or genetic modification of microorganisms to incorporate reporter molecules such as green fluorescent protein (GFP). Commercially available microbe-based biosensors generally require the use of a genetically modified microorganism. For example, KR2017062416A describes a bioreporter microorganism to detect arsenic or cadmium which involves genetic engineering E. coli to express green fluorescent protein or mCherry fluorophore; Dudkowiak, et al. (2011), Int J Thermophys (2011) 32: 762. https://doi.org/10.1007/s10765-010-0852-3 describes possible detection of heavy metal ions using Cyanobacterium by measuring fluorescence of the microorganism at 650 nm based on laser light scattering; Mariscal, et al. (1995), J. Applied Toxicol. Volume 15, Issue 2, March/April 1995, pages 103-107, involves bioassay of toxicity of heavy metals by measuring UV-stimulated fluorescence of E. coli grown in culture medium containing the fluorescent compound 4-methylumbelliferyl-β-D-glucuronide hydrate as a sole carbon source; U.S. Pat. No. 9,976,169 describes a biosensor for detection of arsenic by recombinant E. coli expressing green fluorescent protein; U.S. Patent Publication 2018/0149633A1 also describes a genetically modified microbe expression a fluorescent protein. The use of genetically engineered microbes requires an additional level of complexity as well as raising regulatory issues pertaining to use of genetically modified microbes.

Consequently, there has been a need for a biosensor that employs a wild-type bacterium or bacterial product that does not require genetic modification or chemical modification or chemical agents produced by such wild-type bacteria that can be used to conveniently and economically detect contaminants such as heavy metals.

Accordingly, it is one object of the present disclosure to provide a method, system and biosensor for detecting a heavy metal. The method includes contacting a sample suspected of containing a heavy metal with Bacillus endophyticus or with a fluorescent material isolated from Bacillus endophyticus then identifying the presence of the heavy metal by comparing the sample fluorescence with a control fluorescence. The Bacillus endophyticus has 16s rDNA that is at least 97% identical to that of Bacillus endophyticus DS43.

### BRIEF SUMMARY OF THE INVENTION

The foregoing paragraphs have been provided by way of general introduction, and are not intended to limit the scope of the following claims. The described embodiments, together with further advantages, will be best understood by reference to the following detailed description taken in conjunction with the accompanying drawings.

One aspect of the invention is directed to a method for detecting heavy metals, such as those found in contaminated water and other wastes using a fluorescent material produced from particular strains of Bacillus endophyticus (NCBI:txid135735; genomic sequences incorporated by reference to Jeong, et al., Genome Announc. 2016 May 12; 4(3). pii: e00358-16. doi: 10.1128/genomeA.00358-16), such as wild-type strain DS43 which was isolated from soil in Dammam City, Saudi Arabia. Strain DS43 is distinguishable from other Bacillus strains and may be distinguished from other strains by identified by its partial or full 16s RNA and fluorescent properties upon UV irradiation. Its partial 16s RNA sequence is described by GenBank: KU199806.1 available at hypertext transfer protocol secure://worldwide web.ncbi.nlm.nih.gov/nuccore/KU199806.1.

The inventors have found that fluorescent material is mainly produced under a static condition and exhibits fluorescence at a wavelength of approximately 365 nm under UV radiation.

The invention also pertains to a method for culturing bacteria producing the fluorescent material and to methods for extracting this material for use in detecting heavy metals.

Other related aspects of the invention will be apparent from the following description.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A. Effect of heavy metals on the fluorescent material generated by B. endophyticus DS43. Heavy metals showing an inhibitory effect on the fluorescence. Reaction mixtures contained equal amounts of the fluorescent material and a heavy metal solution. For each metal test: right, heavy metal stock solution; left, reaction mixture.

FIG. 1B. Effect of heavy metals on the fluorescent material generated by B. endophyticus DS43. Heavy metals not showing an inhibitory effect on the fluorescence. Reaction mixtures contained equal amounts of the fluorescent material and a heavy metal solution. For each metal test: right, heavy metal stock solution; left, reaction mixture.

FIG. 2. Depicts daylight illumination (no fluorescence) and UV illumination (yellow fluorescence) of sample using UV transilluminator equipment label: Wisd, WUV-L10 220V UV-transilluminator “wavelengths 365 nm”.

FIG. 3A. Detection of toxic heavy metals using fluorescent material from B. endophyticus DS43 cells supported on chemical substrate in the form of beads.

FIG. 3B. Detection of toxic heavy metals using fluorescent material pre-dissolved in organic solvent and placed in ampule.

FIG. 3C. Detection of heavy metals using an optical biosensor. The major constituent of the biosensor is: a biological recognition element—the fluorescent material and the physical transducer: UV lamp, Reaction chamber, Detector, Amplifier and Display unit.

### DETAILED DESCRIPTION OF THE INVENTION

One embodiment of the invention is a method for detecting a heavy metal that includes contacting a sample suspected of containing a heavy metal with a fluorescent material comprising Bacillus endophyticus or with a fluorescent material isolated from Bacillus endophyticus to form a mixture, irradiating the mixture with electromagnetic radiation suitable for inducing fluorescence, such as ultraviolet or visible light, and selecting a sample containing heavy metal when the fluorescence of the Bacillus endophyticus or the fluorescence of the fluorescent material isolated from Bacillus endophyticus decreases (e.g., is lower) compared to a level of fluorescence in a control sample not containing the heavy metal. In some embodiments fluorescence may decrease by 5, 10, 15, 20, 25, 30, 35, 40, 45, 50, 55, 60, 65, 70, 75, 80, 85, 90, 95, <100% compared to a control or control value.

The fluorescent material is obtainable from Bacillus endophyticus, Bacillus endophyticus strain DS43, or from a strain that has 16s rDNA that is at least 95, 96, 97, 98, 99. 99.5, 99.9 or 100% identical to that of Bacillus endophyticus DS43. A nucleotide sequence encoding 16s rDNA as disclosed herein may have 1, 2, 3, 4, 5, 6, 7, 8, 9, 10 or more deletions, insertions or substitutions of a nucleotide or have at least 80, 90, 95, 99, 99.5, 99.9 or up to 100% sequence identity with the sequences disclosed herein or known rDNA sequences for Bacillus endophyticus. For assessment of variants of strain DS43, the degree of identity between two nucleic acid sequences can be determined using the BLASTn program for nucleic acid sequences, which is available through the National Center for Biotechnology Information (hypertext transfer protocol://_www.ncbi.nlm.nih.gov/blast/Blast.cgi?PAGE=Nucleotides) (last accessed Jul. 31, 2019). The percent identity of two nucleotide sequences may be made using the BLASTn preset “search for short and near exact matches” using a word size of 7 with the filter off, an expect value of 1,000 and match/mismatch of 2/−3, gap costs existence 5, extension 2; or standard nucleotide BLAST using a word size of 11, filter setting “on” (dust) and expect value of 10. In some embodiments, a Bacillus endophyticus strain will have 0, 1, 2, 3, 4, 5, 6, 7, 8, 9, 10, 11, 12, 13, 14, or 15 or more deletions, substitutions or insertions of a nucleotide in its 16s rDNA sequence compared to Bacillus endophyticus DS43 16s rDNA. Further description of the characteristics of Bacillus endophyticus is incorporated by reference to Reva, et al., Int. J. Systematic and Evolutionary Microbiology (2002), 52, 101-107.

A viable stock culture of the Bacillus endophyticus DS43 has been stored in environmental Microbiology Research Laboratory, Environmental Health Department, College of Public Health, Imam Abdulrahman Bin Faisal University. The strain is stored in glycerol at −70° C., with regular subculture on complex media e.g. nutrient agar or tryptone soya medium.

Berekaa et al., Antibiotics sensitivity and heavy metals resistance in PHB-producing bacilli isolated from eastern province, Saudi Arabia Int. J. Agric. Biol. 2016, 18, 1032-1036; investigated the antibiotic profiling, heavy metal resistance and possible toxicity of polyhydroxyalkanoate producing bacteria before large scale production. Among 120 candidate bacterial strains isolated from different soil and sewage water samples screened for PHB production, sixteen bacterial candidates recorded positive results with Sudan Black B and Nile Red A stains. 16S DNA gene analysis revealed high homologies to members of Bacillus cereus group, B. megaterium, B. flexus, B. endophyticus, and B. aryabhattai.

For isolation of DS43 a soil sample was diluted in sterile distilled water and 0.1 mL was plated on nutrient agar (NA plates) with the following composition Peptone 5 g/L; beef 3 g/L; NaCl 5 g/L; and agar 15 g/L. Separate colonies were isolated and further purified. The purified strains were subjected to screening for PHB production after cultivation on modified E2 medium (Berekaa and Al-Thawadi, 2012) and incubated at 37° C. After sterilization of the media, filter-sterilized stain was added (Sudan Black B 0.3 g in 70% Ethanol and Nile Red A stock solution 0.25 g/mL DMSO, use 20 μL to reach final concentration of 0.5 μg/mL).

Interestingly, only B. endophyticus strain DS43 showed clear fluorescence when irradiated with UV after cultivation on complex medium without any dye. Detailed information about the bacterium and molecular identification of the organism is characterized by 16s rDNA analysis and the sequence of the gene deposited in gene bank under the accession number: KU199806 (NIH, NCBI, PubMed, Genbank, Berekaa et al., supra.

The DS43 bacterial strain used in this invention belongs to genus Bacillus endophyticus, and produces a fluorescent material with specific characteristics that are distinguishable from other fluorescent pseudomonads because the fluorescent material produced independent of iron concentration. Generally, pseudomonads either produce fluorescent material depending on iron concentration in medium, thus classified as iron-dependent (produced in medium with limited iron concentration) and iron-independent. Also, the fluorescent material from pseudomonads diffuses in the medium. However, the fluorescent material produced by Bacillus endophyticus DS43 used in this study is independent of iron concentration. Moreover, strain DS43 produces and distributes the fluorescent material both intracellularly and extracellularly. As discovered by the inventors fluorescence from the material produced by strain DS43 disappears after exposure to heavy metals.

While not being bound to any particular hypothesis or explanation, the inventors believe that the inhibition of fluorescence by heavy metals does not involve any enzymatic reactions. This is because such enzymes are denatured during extraction of the fluorescent material from the cells by strong organic solvents (up to 100 vol %) such as acetone, methanol or ethanol. The most probable mechanism for heavy metal detection is the inhibition of fluorescence by physical binding of the fluorescent material with the heavy metal.

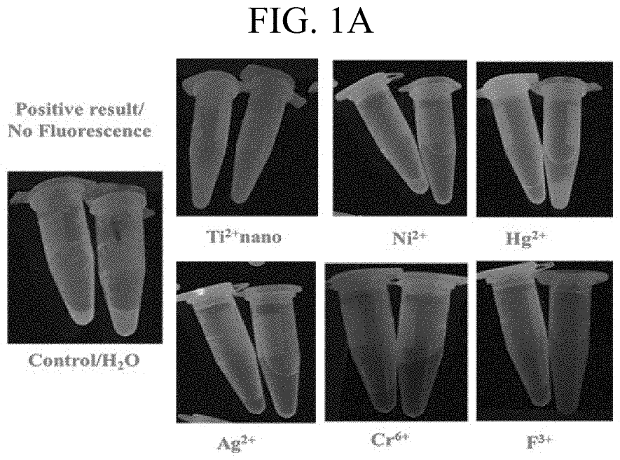

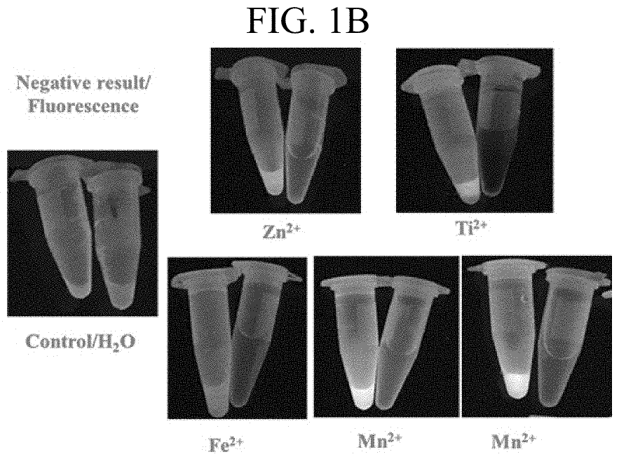

As shown by FIGS. 1A and 1B, the inventors have identified heavy metals that inhibit fluorescence as well as some that do not. To test the inhibitory effect of some metals on the fluorescence generated by the fluorescent material produced by Bacillus endophyticus DS43, a known concentration of heavy metal was mixed with the fluorescent material and the mixture was exposed to UV lamp. Results presented in FIGS. 1A & 1B indicated that the fluorescence is inhibited by salts of some metals, e.g., Co²⁺, TiO₂ nano, Hg²⁺, Cr⁶⁺, Fe³⁺ and Ag²⁺. On the other hand, the fluorescence generated by the material was not inhibited by salts of other metals, e.g. Zn²⁺, TiO₂ salt, Fe²⁺ and Mn²⁺.

Unlike other bacillus strains such as Bacillus megaterium strain, Bacillus endophyticus DS43 produces its pigment without a need for an excess amount of peptone or tryptone during growth on solid medium as well as in liquid medium under different shake conditions. Bacillus endophyticus DS43 can produce the fluorescent material during growth on regular NA medium with 4-fold decrease in peptone concentration that is required by Bacillus megaterium; compare with Magyarosy et al., Appl. Envir. Microbiol. 2002, 68(8), 4095-4101. Also, Bacillus endophyticus DS43 can produce the fluorescent material in LB medium that contains only 1 g of tryptone and 0.5 g yeast extract. However, the fluorescent material produced by Bacillus megaterium strain was only recorded during growth on complex media in presence of excess tryptone, glucose or glycerol; see Magyarosy et al., 2002, supra. In some embodiments, Bacillus endophyticus DS43 is cultured in a medium containing at least 0.1, 0.2, 0.5, 1, 2 or 5 g/L of tryptone or other protein hydrolysate and/or 0.1, 0.2, 0.5, 1, 2 or 5 g/L of yeast extract. Compared to the culture medium described by Magyarosy, et al., supra, the amount of tryptone or other protein hydrolysate, glucose, or glycerol may be reduced 1, 2, 3, 4, 5 or 6-fold.

Detection of heavy metals may be accomplished by forming a mixture by contacting Bacillus endophyticus that contains the fluorescent material with a sample suspected of containing a heavy metal. Representative mixtures include a mixture of Bacillus endophyticus suspended in PBS, saline, Tris, buffer, minimal medium, medium, or other solution with a sample suspected of containing a heavy metal. In some embodiments, the sample suspected of containing the heavy metal may be admixed with viable bacteria in a liquid culture medium or admixed into a solid culture medium, such as into an agar medium that can be streaked with Bacillus endophyticus. Fluorescence of a mixture under irradiation may be measured at the time of mixing or 15, 30, 45, 60 or more minutes after mixing. Fluorescence of bacteria that are streaked on a solid medium may be measured prior to streaking and at various points during the growth of bacteria, such as during lag phase, log or exponential growth phase, stationary phase, or during death phase. Similar procedures may be used with fractions of Bacillus endophyticus or with fluorescent material extracted from Bacillus endophyticus.

In some embodiments of the method, the mixture will be formed and UV irradiated and the fluorescence of the mixture measured compared to a control value, such as a value from the medium prior to addition of the fluorescent material or bacteria producing it, a value taken at the time of mixing a sample suspected of containing a heavy metal with the fluorescent material, or a value taken in the absence of a heavy metal.

In other embodiments, the fluorescent material will be contacted for a specific period of time with the sample to form a mixture, the sample washed off, removed or separated from the fluorescent material, the fluorescent material irradiated and its fluorescence measured compared to a control value. In some embodiments, the control value may be that of the fluorescent material prior to contact with the sample. In some embodiments of this method control samples containing known titrated amounts of a heavy metal may be used to calibrate the results obtained from an unknown sample.

Any sample suspected of containing a heavy metal may be used as-is, optionally diluted or titrated, especially if it is already in a liquid form, or be liquefied or extracted and tested using the methods disclosed herein. A sample used in the method above may be drinking water, graywater, waste water, for example agricultural, industrial, commercial, urban or residential wastewaters, runoff or sewage, and water from mining operations. Water pollution from mining operations includes acid mine drainage, metal contamination of water. Sources include active or abandoned surface and underground mines, processing plants, waste-disposal areas, haulage roads, and tailings ponds. Contamination of water with metals such as Al, As, Ba, Cd, Cr, Cu, Fe, Pb, Mn, Hg, Se, and Ag is a significant problem as high levels of these metals can negatively impact the environment and when ingested cause a variety of medical and health problems. Liquid or liquefied samples can be obtained or prepared from food or drink, animal feeds, or from medical samples, such as from blood, plasma, serum, CSF, synovial fluid, saliva, bile, urine or other biological fluids. The method as disclosed herein may be used in monitoring water quality by detecting presence of heavy metals in drinking water or waste water. It may also be used to detect heavy metal contamination in soils, food products or biological samples.

In a preferred embodiment of the invention, the method and apparatus are used to detect or confirm the presence of mercury in gaseous hydrocarbon streams such as natural gas. Hydrocarbons obtained from certain geologic formations may be contaminated with mercury. Even in the case of hydrocarbon production streams that are produced as gaseous hydrocarbons or gaseous materials obtained from hydrocarbon streams (oil) initially produced in liquid form, mercury may be present in quantities up to 1000 μg/Nm³. The range of mercury may vary broadly in an amount of from 1-500 μg/Nm³, 5-400 μg/Nm³, 10-300 μg/Nm³, 50-200 μg/Nm³ or about 100 μg/Nm³ of gaseous hydrocarbons. The presence of mercury in gaseous hydrocarbons can lead to several significant problems. As already noted above, mercury is a dangerous and undesirable contaminant that may have new neuro toxic effects on humans. In addition, mercury may degrade metals such as aluminum which may be present in gaseous hydrocarbon processing equipment. This mercury is mainly present in its elemental form but may also be present in the form of organic mercury compounds such as dimethyl mercury, diethyl mercury, methyl ethyl mercury or mixed organic/inorganic mercury compounds such as ClHgCH₃.

In a preferred embodiment of the invention the fluorescence of the Bacillus endophyticus is used as a basis for identifying and quantifying the amount of mercury present in a gaseous hydrocarbon stream. The Bacillus endophyticus bacteria or bacterial extract may be contacted with the gaseous hydrocarbon by bubbling the sample through a liquid medium containing the bacteria. The slow decrease and/or disappearance of the fluorescence peak indicates the presence of mercury in the gaseous hydrocarbon stream. The decrease over time as a function of hydrocarbon material that has been bubbled through or otherwise contacted with the bacteria medium may be used to correlate a concentration of mercury present in the gaseous stream. In other embodiments the bacteria or bacterial extract may be contacted with a liquefied form of the gaseous hydrocarbon. In an especially preferred embodiment of the invention the bacteria is directly contacted with the gaseous hydrocarbon in its liquid form, for example, by agitating the a two-phase liquid mixture that includes a liquid form of the gaseous hydrocarbon and an aqueous composition containing Bacillus endophyticus or an extract thereof.

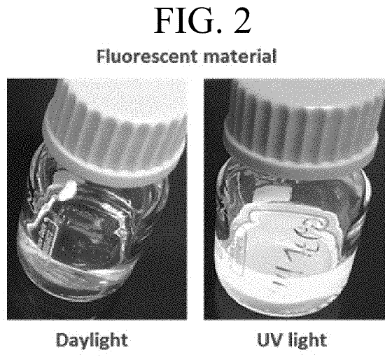

The fluorescent material disclosed herein may be irradiated using ultraviolet light. A typical light source spectrum wavelength ranges for ultraviolet light from 200 to 400 nm—UVC 200 to 280 nm, UVB: 280 to 315 nm, UVA 315 to 400 nm)—for visible light from 400 to 760 nm and for infrared light from 760 to 3000 nm. Preferably, irradiation is performed using ultraviolet light of about 360-370 nm in wavelength. Fluorescence is visible or invisible radiation emitted by certain substances as a result of incident radiation of a shorter wavelength such as X-rays or ultraviolet light. Accordingly, irradiation is typically performed using electromagnetic radiation or light having a shorter wavelength than the light emitted by the fluorescent material. As shown by FIG. 2, visible light is emitted when the material is UV irradiated.

The presence, quantity of, or level of fluorescence may be measured by techniques known in the art including by use of a UV/Vis spectrophotometer, photodiode or phototransistor which converts light into current, a light-dependent resistor (LDR) or a charge couple device (CCD). In some embodiments, the biosensor is further linked to a system containing a processor which can receive and process signals from the above devices and compare them to control values or transmit them via a transmitter or transceiver to a network or to remote devices, such as displays, printers and memory storage devices.

In some embodiments of this method, the sample is suspected of containing a heavy metal that is arsenic, cadmium, chromium, lead or mercury, however, other heavy metals, such as those in the same elemental families as those described herein may also be detected provided they affect the degree of fluorescence of Bacillus endophyticus or a fluorescent material produced by it. In some embodiments of this method the electromagnetic irradiation is ultraviolet irradiation having a wavelength in the range of 360-365-370 which produces fluorescence which can be quantified to detect a decrease in fluorescence caused by presence of a heavy metal.

The decrease in fluorescence may be measured as a function of peak height or integrated peak area. For convenience, the fluorescence determination is preferably made by comparing the peak height of the control sample with the peak height obtained for the sample mixture. The decrease in the peak height may be correlated with increase in the amount or concentration of heavy metal by preparing reference samples which stepwise decrease the amount of heavy metal and hence corresponding fluorescence in the presence of the heavy metal. In another embodiment of the invention the fluorescence or decrease in fluorescence is measured as a function of time. The speed of decrease of fluorescence can be used as a basis for identifying the presence of particular heavy metals in the sample mixture. In other embodiments the peak position (wavelength) can alternately be used as a basis for determining the identity of the heavy metal in the sample.

Preferably the presence of heavy metal has no impact on the viability of the Bacillus endophyticus. The decrease in fluorescence is preferably not due to death or deactivation of biological material but is instead a function of a chemical change or binding of the heavy metal material with the bacteria or chemical components present within the bacterial material.

The decrease in peak height may be 10-95% relative to the peak height of the control sample, for example in the absence of the heavy metal. Preferably changes in peak height vary from 10-90%, 20-80%, 30-70%, 40-60% or about 50% relative to the peak height of the control sample without heavy metal.

In some embodiments of this method the sample is contacted with Bacillus endophyticus or with a fluorescent material produced by this species. For example, the method may be performed using Bacillus endophyticus DS43 or a strain derived by passage or mutation of this strain, for example a strain derived by X-ray or UV irradiation, chemical mutagenesis, or genetic manipulation of Bacillus endophyticus or the DS43 stain. Chemical mutagens include ethylene amine, nitrogen- or sulfur-mustard, diethylnitrosomene, ethylene oxide, diethylsulphonate, and ethylmethane sulfonate as well as other acridines, mustards, nitrosomines, epoxides, or alkylsulphonates. In some instances, directed mutagenesis based on selection of bacteria that have enhanced fluorescence when exposed to UV light, such as UV light having a wavelength of 360-370 nm. In one embodiment, wild-type strain DS43 is passaged 10, 20, 30, 40, 50 or more times to derive a passaged strain with one or more epigenetic or genetic mutations compared to the wild-type or earlier passage of DS43. Preferably, the DS43 may be serially passaged in a medium containing tryptone. A passaged strain of DS43 may produce 1, 2, 3, 4, 5, 6, 7, 8, 9, 10 or more weight % fluorescent compound when grown under the same conditions as the wild-type DS43 strain.

In some embodiments strain DS43 is further modified or mutated, for example, by chemical or radiological mutation or by genetic engineering and recombinant DNA techniques. A modified form of DS43 may have one or more genetic (i.e. to its DNA sequence) or epigenetic changes to its DNA, such as a variant methylation or hydroxymethylation pattern in its genomic DNA, a difference in histone methylation, or difference in microRNA expression, compared to an otherwise identical isolate. Epigenetic variants are those having a heritable phenotype change that does not involve alterations in its DNA sequence.

Typically, wild-type and variant or mutant Bacillus express or synthesize a fluorescent material as disclosed herein, such as a fluorescent material that fluoresces under UV having a wavelength of about 365 nm. In some embodiments a mutant or variant will produce 5, 10, 20, 30, 40, 50, 60, 70, 80, 90, 100 or >100% more fluorescent material than the wild-type strain. Preferably, a wild-type strain is employed as this avoids environmental, biological and regulatory risks associated with genetically engineered microorganisms.

In some embodiments, a wild-type strain such as DS43 can be transformed with exogenous DNA, such as plasmid DNA encoding an antibiotic resistance or other exogenous genes useful in culturing the modified DS43. Antibiotic resistance genes are known in the art and include those for kanamycin, spectinomycin, streptomycin, ampicillin, carbenicillin, bleomycin, erythromycin, polymixin B, tetracycline and chloramphenicol.

In other embodiments, the Bacillus as disclosed herein may be treated with a chemical agent such as a crosslinker like glutaraldehyde or a fixative such as formaldehyde or an alcohol to covalently or non-covalently bind it to a substrate prior to contacting it with a sample suspected of containing a heavy metal. Fixatives, including crosslinking fixatives, precipitating fixatives, oxidizing agents, picrates and HOPE fixative, and fixation methods, including heat fixation and chemical fixation, are known in the art and are incorporated by reference to hypertext transfer protocol secure://en.wikipedia.org/wiki/Fixation_(histology) (last accessed Aug. 19, 2019).

In some embodiments of this method, a fraction or extract of Bacillus endophyticus is used as the fluorescent material, for example, an aqueous or non-aqueous fluorescent organic aromatic compound isolated from Bacillus endophyticus may be employed. Lysed cells, isolated cell membranes or soluble cytoplasmic fractions containing the fluorescent material may be also used.

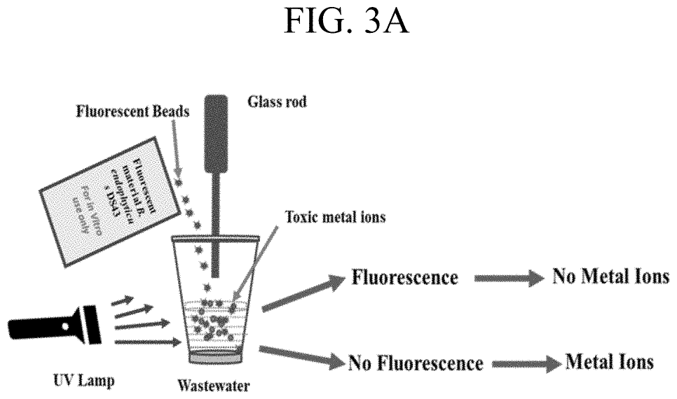

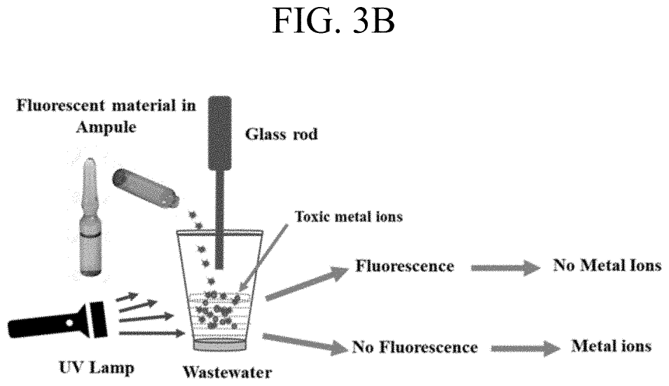

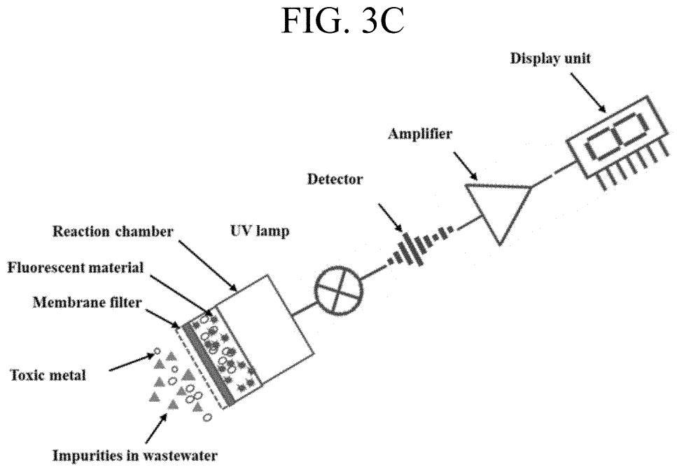

Another embodiment of the invention is directed to an optical biosensor for detecting a target heavy metal in a sample. Such a biosensor includes the fluorescent material, a source of ultraviolet light such as light having a wavelength ranging from 360-370 nm, and a fluorescent light detector; wherein the fluorescent material comprises Bacillus endophyticus or a fluorescent material produced by Bacillus endophyticus. Such a biosensor may comprise one or more of the elements described in FIG. 3A, 3B or 3C.

In some embodiments the fluorescent material in the biosensor is immobilized on a surface of a glass, ceramic, plastic or metal substrate. In some embodiments, the substrate is electrically conductive such as a substrate comprising Fe, Ti, Au, Ag, Cu, Ni, Pt, Pd, Al or stainless steel. In other embodiments, the substrate may comprise a non-conductive material such as polyethylene, polystyrene, polyethylene terephthalate, polycarbonate, polyetheretherketone or polytetrafluoroethene.

The fluorescent material may be immobilized on a surface of a compartment comprising a glass, ceramic, plastic or metal and the optical biosensor can comprise a compartment configured to hold a liquid sample.

A compartment may be a well, such as a microtiter plate well, a tube, channel, conduit, or microfluidic compartment or space, an indentation in a substrate such as in an indented glass slide, or other area designed or configured to hold a liquid sample in contact with the fluorescent material. The fluorescent light detector is configured to receive light from the fluorescent material in the compartment of the biosensor. The detector may directly display a value for the amount of fluorescence or may be operably connected to a processor, such as a computer or smart phone, which processes input from the fluorescent light detection and then outputs the result, for example, on a display, alarm or other output device which indicates or communicates the amount of fluorescence.

In a related embodiment of the invention, the substrate comprising the fluorescent material is placed in contact with a sample suspected of containing a heavy metal and its fluorescence is determined or quantified compared to a control sample or value.

In other embodiments, the fluorescent material is immobilized on or in beads having an average diameter ranging from 1, 2, 5, 10, 20, 50, 100, 200, 500 to 1,000 μm or in some cases, having an average diameter of 1, 2, 5, 10, 20, 50, 100, 200, 500 to 1,000 nm. These ranges include all intermediate subranges and values. For example, the fluorescent material can be immobilized on or in beads having an average diameter ranging from 1 nm to 1,000 μm; the optical biosensor can further include a compartment, such as a microtiter plate well, a tube, a bottle or other container suitable to hold a liquid sample in contact with said beads. The compartment or biosensor may further comprise a stirrer, vibrator, or other agitator that facilitates contact between the beads and a liquid sample. The fluorescent light detector is configured to receive light from the fluorescent material on the beads in the biosensor. The fluorescent light detector is configured to receive light from the fluorescent material in the compartment of the biosensor. The detector may directly display a value for the amount of fluorescence or may be operably connected to a processor, such as a compute or smart phone, which processes input from the fluorescent light detection and then outputs the result, for example, on a display, alarm or other output device which indicates or communicates the amount of fluorescence.

In a related embodiment of the invention, the beads to which the fluorescent material is attached are placed in contact with a sample suspected of containing a heavy metal and their fluorescence is determined or quantified by the fluorescent light detector compared to a control sample or value.

In some embodiments, the fluorescent material after contact with a heavy metal may be regenerated by removing heavy metal bound or otherwise associated with the material, for example, by washing in a solution at a pH less than 5, 6, 7, washing in a solution at a pH of at least 7, 8 or 9, or by washing, or by washing with or eluting with various concentrations of saline or with a solution containing a chelating agent like EDTA (ethylenediaminetetraacetic acid), EGTA (ethylene glycol-bis(β-aminoethyl ether), DMPS (2,3-dimercapto-1-propanesulfonic acid sodium), or DMSA (Meso-2,3-dimercaptosuccinic acid).

In some embodiments where fluorescence of viable Bacillus endophyticus is used to detect heavy metals in a sample, the bacteria, which may be in solution or fixed to a substrate, may be allowed to regenerate in a nutrient medium, for example, a substrate comprising Bacillus endophyticus once used to detect a heavy metal, may be washed to remove residual traces of heavy metals and then placed in a culture medium to permit growth or regeneration of viable bacteria.

In one embodiment, the fluorescent material after contact with a heavy metal or other compound that eliminates or reduces its fluorescence is contacted with a chelating agent such as EDTA (ethylenediaminetetraacetic acid), EGTA (ethylene glycol-bis(β-aminoethyl ether), DMPS (2,3-dimercapto-1-propanesulfonic acid sodium), or DMSA (Meso-2,3-dimercaptosuccinic acid) in order to restore or regenerate fluorescent activity.

Another embodiment of the invention is directed to a method for producing Bacillus endophyticus that contains a material that when irradiated with ultraviolet light fluoresces under UV light having a wavelength of 360 to 370 nm, comprising culturing Bacillus endophyticus at a pH ranging from pH 6.0, 6.2, 6.4, 6.6, 6.8, 7.0, 7.2, 7.4, 7.6, 7.8 to 8.0, at a temperature ranging from 10, 15, 20, 25, 30, 35, 40 to 45° C., and in a medium lacking one, more than one, or all heavy metals; exposing a sample of the culture Bacillus endophyticus to ultraviolet light and measuring an intensity of visible light fluorescence at a wavelength that is longer than that of the UV irradiation, and harvesting the Bacillus endophyticus when a predetermined degree of fluorescence is obtained. The predetermined degree of fluorescence may represent the maximal fluorescence obtained for a particular culture of bacteria. Thus, bacteria may be harvested once maximal fluorescence has been reached and begins to decline. When irradiated with UV, the fluorescent material produced by B. endophyticus DS43 can be visibly detected as shown by FIG. 2.

In some embodiments of this method the Bacillus endophyticus is cultured in a medium that contains a carbon or nitrogen source other than starch, casein or gelatin; in a medium containing citrate or gluconate; in a medium containing at least one of L-arabinose, D-glucose, meso-inositol, D-mannitol, D-mannose, melibiose, D-rhamnose, ribose or sucrose; and/or in a medium containing less than 10 wt % NaCl. In other embodiments of this method the Bacillus endophyticus is cultured under microaerophilic conditions under a concentration of oxygen ranging from 0, >0, 1, 2, 3, 4, 5, 6, 7, 8, 9, 10, 11, 12, 13, 14, 15, 16, 17, 18, 19, 20 or <21%. In some embodiments, the bacteria may be cultured in a medium containing antioxidants, such as cysteine, mercaptoethanol, DTT, glutathione, catechin hydrate, quercetin dehydrate, chlorogenic acid, vitamin C, or vitamin E.

In some embodiments, this Bacillus endophyticus disclosed herein may be cultured at a temperature ranging from 20−40° C., for about 24-72 hours, using an inoculum size of 0.5 to 1.5% v/v, at various pHs including 3, 4, 5, 6, 7, 8, 9 or 10. They may be cultured using various carbon sources including glucose, sucrose, fructose, maltose or the most common and cheap raw material in Saudi Arabia the date syrup or DEBS; or various nitrogen sources such as tryptone, beef extract, peptone, ammonium salts, nitrate salts which may be present at a final conc. 0.05, 0.1, 0.15, 0.2, 0.25, 0.3 wt % or greater, they may contain various amino acids, such as cysteine, leucine, methionine, tryptophan, histidine, glutamine and proline typically at a final concentration in the culture medium of 0.05, 0.1, 0.15, 0.2, 0.25, 0.3 wt % or greater.

Suitable media include nutrient broth, tryptone soya broth or tryptic soy broth (TSB). Cultivation may be static or under agitation such as shaking an 80, 100, 120, 150-200 rpm. In some embodiments, media described by Garrision, Earl Raymond, “The fluorescent bacteria in dairy products” (1940). Retrospective Theses and Dissertations, hypertext transfer protocol secure://lib.dr.iastate.edu/rtd/13675 (last accessed Nov. 30, 3030, incorporated by reference) may be used. However, few media are useful for growth of and enhanced fluorescent production. These include beef extract-peptone agar medium, beef extract-tryptone-dextrose-skim milk agar, and tryptone agar medium which produces more fluorescence material than beef extract-peptone medium. Supplements such as magnesium sulfate (e.g., 0.05-0.5% g/L) or K₂HPO₄ may be added to either beef extract-peptone agar or to beef extract-tryptone-dextrose-skim milk agar medium.

In some embodiments, the fluorescent compound is extracted from the cells by the use of an organic solvent or using organic solvents with different polarities such as acetone, methanol or ethanol. Cells may be lysed prior to extraction.

In some embodiments, an extract will contain a water-soluble fluorescent pigment that is easily combined with contaminated water or aqueous samples containing heavy metals. In other embodiments, a fluorescent pigment may partition into a solvent more hydrophobic than water or stay associated with a membrane or solid fraction of Bacillus endophyticus.

The method disclosed herein may further include disrupting the harvested Bacillus endophyticus, separating the disrupted Bacillus endophyticus into a solid and soluble fraction, exposing the fractions to ultraviolet light and selecting a fraction that fluoresces when exposed to light having a wavelength of 360 to 370 nm.

Means for disrupting bacteria are known in the art and include, but are not limited to sonication, French pressing, homogenizer treatment, microfluidization, bead beating, cryopulverization, nitrogen decompression (or decompression with carbon dioxide, nitrous oxide, carbon monoxide or other gases), osmotic shock, and enzyme treatment. During decompression nitrogen gas is preferred because it is non-reactive and does not alter the pH of the disrupted cells and can provide a more uniform disrupted product than other modes of disruption. Solid and liquid fractions of disrupted cells may be further separated by filtration or centrifugation or by other methods known in the art. Water soluble and hydrophobic fractions of disrupted cells may be separated by extraction with organic or aqueous solvents. Common extractants include ethyl acetate<acetone<ethanol<methanol<acetone:water (7:3)<ethanol:water (8:2)<methanol:water (8:2)<water) in increasing order of polarity according to the Hildebrand solubility parameter. Such solvents include n-pentane, n-hexane, methanol, ethanol, propanol, butanol, diethyl ether, ethyl acetate, chloroform, dichloromethane, and acetone.

Once cells are disrupted or once an extract is produced, it may be refrigerated, frozen, dried or kept under an inert atmosphere not containing oxygen. An extract can be reduced to a dried form using a centrifugal evaporator or a freeze-drier.

In some embodiments, the invention is directed to a composition comprising intact, living Bacillus endophyticus, membranes or other solid components of Bacillus endophyticus, or comprising cytosol of other soluble components of Bacillus endophyticus produced by the method disclosed herein that when irradiated with ultraviolet or visible light fluoresce at 360 to 370 nm.

Use of the fluorescent material conveniently obtained from Bacillus endophyticus renders other analytic methods used to detect heavy metal ions unnecessary. Such methods include those which use CdSe or other types of quantum dots, sol-gel methods, bioreporter systems including those expressing green fluorescent protein, mCherry, or other reporter genes or recombinant DNA procedures, inhibition of urease activity or any other enzymatic activity, use of acridine orange dye, rhodamine 6G fluorescence compounds, or any external dye, microfluidic devices, or other microorganisms such as Escherichia coli or Cyanobacterium Synechocystis aquatilis.

Heavy metal pollutants include, but are not limited to, aluminum, antimony, arsenic, barium, bismuth, cadmium, lead, mercury, nickel, tin and uranium. The fluorescence of the compound is inhibited in presence of heavy metals. Sources of heavy metal pollutants are metal mining, metal smelting, metallurgical industries, and other metal-using industries, waste disposal, corrosions of metals in use, agriculture and forestry, forestry, fossil fuel combustion, and sports and leisure activities. Chromium, arsenic, cadmium, mercury, and lead have the greatest potential to cause harm on account of their extensive use, the toxicity of some of their combined or elemental forms, and their widespread distribution in the environment. Hexavalent chromium, for example, is highly toxic as are mercury vapor and many mercury compounds. These five elements have a strong affinity for sulfur; in the human body they usually bind, via thiol groups (—SH), to enzymes responsible for controlling the speed of metabolic reactions. The resulting sulfur-metal bonds inhibit the proper functioning of the enzymes involved; human health deteriorates, sometimes fatally. Chromium (especially in its hexavalent form) and arsenic are carcinogens; cadmium causes a degenerative bone disease; and mercury and lead damage the central nervous system. Description of these and other heavy metals is incorporated by reference to hypertext transfer protocol secur://en.wikipedia.org/wiki/Heavy_metals (last accessed Aug. 14, 2019). In some embodiments, the method as disclosed herein can detect heavy metals in concentrations of at least 0.1, 0.5, 1, 2, 5, 10, 20, 30, 40, 50, 100, 200 ppm or more. In some embodiments, the method disclosed herein will detect concentrations of arsenic that are 5.0 ppm or more, barium that are 100 ppm or more, cadmium that are 1.0 ppm or more, chromium that are 5.0 ppm or more, lead that are 5.0 ppm or more, mercury that are 0.2 ppm or more, selenium that are 1.0 ppm or more or silver that are 5.0 ppm or more.

Heavy metal testing as disclosed herein may be used to detect, assay, determine levels of heavy metals in sample including in industrial, mining, medical wastes, dental wastes, wastewater, sewage, or water runoff including seepage from underground sources or from landfills, or in foods or biological samples. It may be used to monitor water quality, soil contamination or to assess quality of a soil. It may also be used to determine heavy metal burden in humans and other animals, including in biological samples obtained from a subject such as urine, plasma, serum or blood. Food, such as fish, shellfish, meats and vegetable or grain products, as well as water and soil, may be tested by contacting sample quantities or samples that have been suspended in an aqueous medium or other solute. It may also be used to detect or assess nutritional deficiencies, gastrointestinal function, hepatic detoxification, metabolic abnormalities, and diseases of environmental origin.

In some embodiments, an entire bacterium may be used as part of a biosensor for heavy metals. In other embodiments, a fraction of a bacterium that contains the fluorescent material, such as a membrane fraction or cytosol fraction, may be used. In still other embodiments, the fluorescent material is substantially removed from 10, 20, 30, 40, 50, 60, 70, 80, 90, 95, <100 or 100% by weight other components in which it is present in a bacterium, for example, the fluorescent material may be purified away from other cellular components by size exclusion, ion exchange or affinity chromatography.

In some embodiments, the fluorescent material is refined or concentrated so that it exhibits at least 1.1, 1.2, 1.5, 2, 3, 4, 5 or >5 times more fluorescence than an equal weight or equal volume of unrefined material, such as unrefined living or disrupted Bacillus endophyticus DS43 cells. Preferably, the isolated cells or disrupted or fractionated material is suspended in a buffer than maintains the fluorescent activity, such as a buffer that does not contain heavy metals or other materials that diminish the fluorescence at 360 to 370 nm. Alternatively, it may be purified by separation into a hydrophilic or hydrophobic fraction or by methods for purification of organic compounds, such as by sublimation, crystallization, distillation, differential extraction or chromatography.

In one embodiment, Bacillus endophyticus may be grown on a solid or in a liquid medium at 25° C. or at an elevated temperature up to 45° C., removed from a solid medium by scrapping or from a liquid medium by filtration or centrifugation, optionally disrupted by sonication, sheering, freezing and thawing or other disruption method, extracted with a solvent such as acetone, a butanone, or another ketone or methyl acetate or ethyl acetate, filtered to separate pulp and filtrate, the pulp may be again extracted as described above and refiltered, the filtrates may be combined and concentrated, for example, by removing solvent under a vacuum until a precipitate forms, the insoluble precipitate may be washed in the solvent described above or in another solvent to remove residual soluble material, and solid fluorescent material recovered by removing residual solvent(s).

The fluorescent material may be incorporated onto or into a substrate, into a porous substrate, or into a liquid for use in detecting heavy metals.

### Example 1

Method for Detecting a Heavy Metal Using Intact or Fractionated Bacillus endophyticus DS43

Bacillus endophyticus DS43 cells are grown to log-phase at 25° C. on tryptone soya broth peptide-meat extract medium, harvested by centrifugation and washed with phosphate buffered saline 2 times and then resuspended in PBS at pH 7.4 at a concentration of 5×10⁸ cells/ml at an OD₆₀₀ of 1.0. Half of the washed and resuspended Bacillus endophyticus DS43 cells are sonicated on ice 3 times for 30 seconds. The sonicated cells are centrifuged at 15,000×g for 10 mins and the pellet and soluble fractions separated. The pellet and soluble fractions are resuspended to the original volume of the sonicated cells in PBS.

Samples of the non-sonicated cells, the resuspended pellet, and the reconstituted soluble fraction were titrated with zero (control) and increasing concentrations of soluble arsenic, chromium, lead and mercury and then exposed to UV light having a wavelength of 365 nm. The fluorescence of each sample is measured using a spectrophotometer and compared to the control value (0% added heavy metal). Typically, cell concentration ranges from 10⁶ to 10⁸ CFU/mL and cells are harvested for tests during exponential phase (e.g, after 48 to 72 hours, preferably 48 hours for medium described above).

### Example 2

Method for Detecting a Heavy Metal Using Intact Bacillus endophyticus DS43 Bound to a Substrate

Bacillus endophyticus DS43 cells are grown to log-phase on tryptone soya broth or peptide-meat extract medium, harvested by centrifugation and washed 2 times and then resuspended in phosphate buffered saline (PBS) at pH 7.4 at a concentration of 5×10⁸ cells/ml or an OD₆₀₀ of 1.0. Then, 50 μl of washed cells were placed into each well of a plastic 96-well flat bottom microtiter plate. 150 μl containing zero (control) and increasing concentrations of soluble arsenic, chromium, lead and mercury were added to the wells. Fluorescence under UV light of 365 nm wavelength was measured at 0, 15, 30 and 60 minutes and compared to the control value (0% added heavy metals). Typically, cell concentration ranges from 10⁶ to 10⁸ CFU/mL and cells are harvested for tests during exponential phase (e.g, after 48 to 72 hours, preferably 48 hours for medium described above).

### Example 3

Method for Detecting a Heavy Metal Using Intact Bacillus endophyticus DS43 Grown on an Agar Medium

Plates (35 mm in diameter) containing an agar tryptone soya agar or peptide-meat extract agar medium (15 wt % agar) the concentrations of soluble As, Cr, Hg and Pb of 0.05, 0.1, 0.5, 1.0, 5.0, 10.0 and 50.0 mg/l are prepared. Bacillus endophyticus DS43 cells (200 μl of a 10⁸ CFU/ml exponentially growing culture) are uniformly plated on the agar and cultured at 25° C. overnight to form bacterial lawns or evenly dispersed colonies. For the grown bacterial lawns only, the fluorescence of a unit section of each lawn is measured under irradiation by UV light having wavelength of 365 nm. The concentration of each heavy metal is correlated with the degree of fluorescence. Typically, cell concentration ranges from 10⁶ to 10⁸ CFU/mL and cells are harvested for tests during exponential phase (e.g, after 48 to 72 hours, preferably 48 hours for medium described above).

### Example 4

Extraction of Fluorescent Material from Bacillus endophyticus DS43

Bacillus endophyticus strain DS43 cells were cultivated on tryptone soya agar (TSA) medium for 48 hours. At the end of incubation period, cells were collected from the surface of TSA plates by suspension in sterile distilled water. Subsequently, they separated from suspending liquids by centrifugation at 7500 rpm for 10 min, washed with distilled water, and re-centrifuged at the same condition. The fluorescent material was extracted by suspending cells in acetone and regular vortex every 15 min for 2 to 4 hours at room temperature. Finally, cell debris separated by centrifugation at 10,000 rpm for 10 min, and the acetone extracted fluorescent material was concentrated by evaporation at room temperature. To detect some heavy metals, samples of the fluorescent material were incubated with increased concentrations of some heavy metals then exposed to UV light having a wavelength of 365 nm. The fluorescence of each sample is measured using a spectrophotometer and compared to the control value (0% added heavy metal).

1. A method for detecting a heavy metal comprising:
contacting a sample with Bacillus endophyticus or with a fluorescent material isolated from Bacillus endophyticus to form a mixture,
irradiating the mixture with ultraviolet or visible light, and
identifying the presence of the heavy metal based on a sample fluorescence of the mixture by comparing the sample fluorescence with a control fluorescence from a control sample not containing the heavy metal; wherein the sample fluorescence and the control fluorescence are from the Bacillus endophyticus or the fluorescent material isolated from Bacillus endophyticus;
wherein said Bacillus endophyticus has 16s rDNA that is at least 97% identical to that of Bacillus endophyticus DS43.

2. The method of claim 1, wherein the sample is contacted with Bacillus endophyticus.

3. The method of claim 1, wherein Bacillus is Bacillus endophyticus DS43.

4. The method of claim 1, wherein the sample is contacted with the fluorescent material isolated from Bacillus endophyticus which is a fluorescent organic aromatic compound.

5. The method of claim 1, wherein the heavy metal is arsenic.

6. The method of claim 1, wherein the heavy metal is cadmium.

7. The method of claim 1, wherein the heavy metal is chromium.

8. The method of claim 1, wherein the heavy metal is lead.

9. The method of claim 1, wherein the heavy metal is mercury.

10. The method of claim 1, wherein the irradiating is with ultraviolet light at a wavelength of 360 to 370 nm.

11. An optical biosensor for detecting a target heavy metal in a sample, the biosensor comprising:
a fluorescent material,
a source of ultraviolet or visible light, and
a detector of fluorescence at a wavelength ranging from 360 to 370 nm;
wherein the fluorescent material comprises Bacillus endophyticus or a fluorescent material produced by Bacillus endophyticus.

12. The optical biosensor of claim 11, wherein the fluorescent material is immobilized on a surface of a glass, ceramic, plastic or metal substrate.

13. The optical biosensor of claim 11, wherein the fluorescent material is immobilized on a surface of a compartment comprising a glass, ceramic, plastic or metal substrate and wherein said compartment is configured to hold a liquid sample.

14. The optical biosensor of claim 11, wherein the fluorescent material is immobilized on or in beads having an average diameter ranging from 1 to 1,000 μm.

15. The optical biosensor of claim 11, wherein the fluorescent material is immobilized on or in beads having an average diameter ranging from 1 to 1,000 μm, and wherein said optical biosensor further comprises a compartment suitable for contain a liquid sample and said beads.

16. A method for producing Bacillus endophyticus that contains a material that when irradiated with ultraviolet light fluoresces at 360 to 370 nm, comprising:
culturing Bacillus endophyticus at a pH ranging from pH 6.0 to 8.0, at a temperature ranging from 10 to 45° C., and in a medium lacking heavy metals;
exposing a sample of the culture Bacillus endophyticus to ultraviolet light or visible light and measuring an intensity of fluorescence at 360 to 370 nm, and
harvesting the Bacillus endophyticus when a predetermined degree of fluorescence at 360 to 370 nm is obtained.

17. The method of claim 16, wherein the Bacillus endophyticus is cultured in a medium that contains a carbon or nitrogen source other than starch, casein or gelatin; in a medium containing citrate or gluconate; in a medium containing at least one of L-arabinose, D-glucose, meso-inositol, D-mannitol, D-mannose, melibiose, D-rhamnose, ribose or sucrose; and/or in a medium containing less than 10 wt % NaCl

18. The method of claim 16, wherein the Bacillus endophyticus is cultured under microaerophilic conditions under a concentration of oxygen ranging from 2 to 10%.

19. The method of claim 16, further comprising disrupting the harvested Bacillus endophyticus, separating the disrupted Bacillus endophyticus into a solid and soluble fraction, exposing the fractions to ultraviolet light and selecting a fraction that fluoresces under irradiation at 360 to 370 nm.

20. A composition comprising intact, living Bacillus endophyticus, membranes or other solid components of Bacillus endophyticus, or comprising cytosol of other soluble components of Bacillus endophyticus produced by the method of claim 16 that when irradiated with ultraviolet or visible light fluoresce under irradiation at 360 to 370 nm.
